SEQUENCE LISTING (2 sequences)

SEQ ID NO: 1 - protein, 494 aa, Lepisosteus oculatus
MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

SEQ ID NO: 2 - protein, 355 aa, Artificial Sequence
  Truncated sequence
TVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

(19) United States
(12) Patent Application Publication
(10) Pub. No.: US 2023/0175030 A1
(43) Pub. Date: Jun. 8, 2023

(54) NUCLEIC ACID POLYMER WITH AMINE-MASKED BASES

(71) Applicant: Nuclera Nucleics Ltd., Cambridge (GB)

(72) Inventors: Michael Chun Hao Chen, Cambridge (GB); Gordon Ross McInroy, Cambridge (GB); Martin Edward Fox, Cambridge (GB); Michal Robert Matuszewski, Cambridge (GB)

(21) Appl. No.: 17/610,900

(22) PCT Filed: May 14, 2020
(86) PCT No.: PCT/GB2020/051181

§ 371 Date: Nov. 12, 2021

(30) Foreign Application Priority Data
May 14, 2019 (GB) 1906772.7

(51) Int. Cl. C12P 19/34; C12P 19/18; C07H 21/04

(52) U.S. Cl. CPC C12P 19/34; C12P 19/18; C07H 21/04

(57) ABSTRACT

Disclosed an improved process for synthesising a nucleic acid strand using cycles of template independent enzyme extension. Having one or more of the amino groups on the base heterocyclic groups masked with protecting groups helps to prevent secondary structure in the extended strand, thereby improving access of the enzyme to the 3′ OH terminus for extension.

### RELATED APPLICATIONS

This application is a 35 U.S.C. § 371 national stage filing of International Application No. PCT/GB2020/051181, filed May 14, 2020, which claims the benefit of priority to United Kingdom Patent Application No. 1906772.7, filed May 14, 2019, the contents of each of which are herein incorporated by reference in their entirety.

### FIELD OF THE INVENTION

The invention relates to nucleic acid polymers having one or more of the amino groups on the base heterocyclic groups masked with protecting groups. The invention also relates to a method of producing said polymers.

### BACKGROUND TO THE INVENTION

Nucleic acid synthesis is vital to modern biotechnology. The rapid pace of development in the biotechnology arena has been made possible by the scientific community's ability to artificially synthesise DNA, RNA and proteins.

Artificial DNA synthesis allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. Despite being a mature technology, it is highly challenging to synthesise a DNA strand greater than 200 nucleotides in length in viable yield, and most DNA synthesis companies only offer up to 120 nucleotides routinely. In comparison, an average protein-coding gene is of the order of 2000-3000 contiguous nucleotides, a chromosome is at least a million contiguous nucleotides in length and an average eukaryotic genome numbers in the billions of nucleotides. In order to prepare nucleic acid strands thousands of base pairs in length, all major gene synthesis companies today rely on variations of a ‘synthesise and stitch’ technique, where overlapping 40-60-mer fragments are synthesised and stitched together by enzymatic copying and extension. Current methods generally allow up to 3 kb in length for routine production.

The reason DNA cannot be synthesised beyond 120-200 nucleotides at a time is due to the current methodology for generating DNA, which uses synthetic chemistry (i.e., phosphoramidite technology) to couple a nucleotide one at a time to make DNA. Even if the efficiency of each nucleotide-coupling step is 99% efficient, it is mathematically impossible to synthesise DNA longer than 200 nucleotides in acceptable yields. The Venter Institute illustrated this laborious process by spending 4 years and 20 million USD to synthesise the relatively small genome of a bacterium.

Known methods of DNA sequencing use template-dependent DNA polymerases to add 3′-reversibly terminated nucleotides to a growing double-stranded substrate. In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

Various attempts have been made to use a terminal deoxynucleotidyl transferase for de novo single-stranded DNA synthesis. Uncontrolled de novo single stranded DNA synthesis, as opposed to controlled, takes advantage of TdT's deoxynucleoside triphosphate (dNTP) 3′ tailing properties on single-stranded DNA to create, for example, homopolymeric adaptor sequences for next-generation sequencing library preparation. In controlled extensions, a reversible deoxynucleoside triphosphate termination technology needs to be employed to prevent uncontrolled addition of dNTPs to the 3′-end of a growing DNA strand. The development of a controlled single-stranded DNA synthesis process through TdT would be invaluable to in situ DNA synthesis for gene assembly or hybridization microarrays as it removes the need for an anhydrous environment and allows the use of various polymers incompatible with organic solvents.

However, TdT has not been shown to efficiently add nucleoside triphosphates containing 3′ reversibly terminating moieties for building up a nascent single-stranded DNA chain necessary for a de novo synthesis cycle. A 3′-O— reversible terminating moiety would prevent a terminal transferase like TdT from catalysing the nucleotide transferase reaction between the 3′-end of a growing DNA strand and the 5′-triphosphate of an incoming nucleoside triphosphate.

There is therefore a need to identify modified terminal deoxynucleotidyl transferases that readily incorporate 3′-O— reversibly terminated nucleotides. Said modified terminal deoxynucleotidyl transferases can be used to incorporate 3′-O— reversibly terminated nucleotides in a fashion useful for biotechnology and single-stranded DNA synthesis processes in order to provide an improved method of nucleic acid synthesis that is able to overcome the problems associated with currently available methods.

The inventors of the current invention have identified modified TdTs that readily incorporate 3′-O— reversibly terminated nucleotides, as disclosed in patent application GB1901501.5 and PCT/G B2020/050247.

The single stranded nucleic acid polymers produced using said method of template-independent synthesis are susceptible to the formation of secondary structures which inhibit access of said TdT enzymes to the 3′OH terminus for extension.

The inventors have previously discovered certain modified nucleotides can be incorporated using terminal transferases. Modified nucleotides suitable for terminal transferase extension have been disclosed in for example PCT/GB2018/053305. However the inventors have appreciated that certain combinations of nucleotides having N-protected bases can be used to make nucleic acid polymers in which a subset of the bases is N-protected.

The inventors have found that having one or more of the amino groups on the base heterocyclic groups masked with protecting groups helps to prevent secondary structure in the extended strand, thereby improving access of the enzyme to the 3′OH terminus for extension.

The protecting groups on the amino groups can be readily removed at the end of the synthesis reaction.

### SUMMARY

The invention relates to nucleic acid polymers having one or more of the amino groups on the base heterocyclic groups masked with protecting groups. The invention also relates to a method of producing said polymers. The polymer can have a portion of the base amine groups masked. The portion may be 100% of one type of base (i.e. all the amino groups on the G, or A or C bases in the polymer may be masked) or the portion may be less than 100%.

Disclosed is a single stranded nucleic acid polymer with a 3′ OH end having all of at least one type of nitrogenous heterocycle amine masked, wherein the amine masking reduces the formation of secondary structure.

Disclosed is a single stranded nucleic acid polymer with a 3′-O-reversibly terminated 3′-end having all of at least one type of nitrogenous heterocycle amine masked, wherein the amine masking reduces the formation of secondary structure.

Disclosed is a single stranded nucleic acid polymer with a 3′O—NH₂ end having all of at least one type of nitrogenous heterocycle amine masked, wherein the amine masking reduces the formation of secondary structure.

Disclosed is a single stranded nucleic acid polymer with a 3′ O—CH₂N₃ end having all of at least one type of nitrogenous heterocycle amine masked, wherein the amine masking reduces the formation of secondary structure.

Disclosed is a method of nucleic acid synthesis comprising:
(a) providing an initiator sequence;
(b) adding extension reagents comprising a 3′-O-reversibly terminated nucleoside triphosphate having an amine-masked nitrogenous heterocycle and a terminal deoxynucleotidyl transferase (TdT) to said initiator sequence to add a single nucleotide to the initiator sequence;
(c) removal of the extension reagents;
(d) cleaving the 3′-O blocking group from the extended nucleic acid polymer;
(e) adding extension reagents comprising a 3′-O-reversibly terminated nucleoside triphosphate having nitrogenous heterocycles with free amine groups and a terminal deoxynucleotidyl transferase (TdT) to said initiator sequence to add a single nucleotide to the initiator sequence;
(f) removal of the extension reagents;
(g) cleaving the 3′-O blocking group from the extended nucleic acid polymer;
(h) repeating steps (b)-(g) to produce an extended nucleic acid polymer having a subset of the amine groups masked amine-masked nitrogenous heterocycles; and
(i) releasing the NH₂ groups on the nitrogenous heterocycles by removing the amine-masks.

Disclosed is a method of nucleic acid synthesis comprising:
(a) providing an initiator sequence;
(b) adding extension reagents comprising a 3′-O—NH₂ blocked or a 3′—O—CH₂N₃ blocked nucleoside triphosphate having an amine-masked nitrogenous heterocycle and a terminal deoxynucleotidyl transferase (TdT) to said initiator sequence to add a single nucleotide to the initiator sequence;
(c) removal of the extension reagents;
(d) cleaving the 3′-O blocking group from the extended nucleic acid polymer;
(e) adding extension reagents comprising a 3′-O—NH₂ blocked or a 3′—O—CH₂N₃ blocked nucleoside triphosphate having nitrogenous heterocycles with free amine groups and a terminal deoxynucleotidyl transferase (TdT) to said initiator sequence to add a single nucleotide to the initiator sequence;
(f) removal of the extension reagents;
(g) cleaving the 3′-O blocking group from the extended nucleic acid polymer;
(h) repeating steps (b)-(g) to produce an extended nucleic acid polymer having a subset of the amine groups masked amine-masked nitrogenous heterocycles; and
(i) releasing the NH₂ groups on the nitrogenous heterocycles by removing the amine-masks.

Disclosed is a single stranded nucleic acid polymer comprising formula (I):

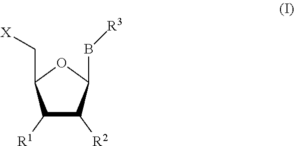

wherein:

R¹ represents O-azidomethyl, aminooxy, O-allyl group, O-cyanoethyl, O-acetyl, O-nitrate, O-phosphate, O-acetyl levulinic ester, O-tert butyl dimethyl silane, -trimethyl(silyl)ethoxymethyl, O-ortho-nitrobenzyl, or O-para-nitrobenzyl.

R² represents —H, or —OH

X represents a single stranded nucleic acid polymer having all of at least one type of nitrogenous heterocycle amine masked, wherein the amine masking reduces the formation of secondary structure;

R³ represents an amine masking group; and

B represents a nitrogenous heterocycle.

Disclosed is a single stranded nucleic acid polymer comprising formula (I):

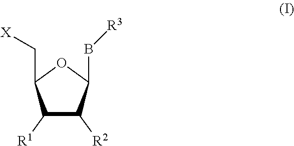

wherein:

R¹ represents —OH, —ONH₂, or —OCH₂N₃;

R² represents —H, or —OH

X represents a single stranded nucleic acid polymer having all of at least one type of nitrogenous heterocycle amine masked, wherein the amine masking reduces the formation of secondary structure;

R³ represents an amine masking group; and

B represents a nitrogenous heterocycle.

When R¹ represents —OCH₂N₃, R³ cannot be —N₃, such that the two groups are orthogonal.

### BRIEF DESCRIPTION OF THE FIGURES

FIG. 1. Engineered TdTs are capable of incorporation of amine-masked nitrogenous base triphosphates to form amine-masked nucleic acid polymers. Shown above are examples of TdT-catalyzed addition of amine-masked nucleoside triphosphates to form amine masked nucleic acid polymers. In the left hand example, the amine-masked 4-azido-5-methyl-2′-deoxy-3′-aminoxy-cytidine 5′-triphosphate is added to a nucleic acid of length N (see left gel, N versus N+1) by TdT. N* is a 3′-phosphorylated version of N. In the right hand example, 6-azido-2′-deoxyadenosine 5′-triphosphate is added to a nucleic acid of length N (see right gel, N versus N+1) by TdT. By virtue of the unblocked 3′-hydroxyl, a tail of 6-azido-2′-deoxyadenosine is made to form an amine-masked nucleic acid polymer. All reactions were run in an appropriate buffer containing the nucleoside 5′-triphosphate (0.5 mM), inorganic pyrophosphatase, engineered TdT, and divalent salts. Reactions were analyzed by denaturing PAGE and visualized by SybrGold staining.

FIG. 1 above demonstrates that TdT is capable of performing both controlled and uncontrolled enzymatic DNA synthesis utilizing amine-masked nucleotides.

FIG. 2. Amine-masked nucleic acid polymers do not support secondary structure duplex formation. In this experiment, 5′-biotynlated oligonucleotide homopolymers were immobilized on a neutravidin plate (High Binding Capacity 96-well Strips, Thermo Fisher Scientific). These oligonucleotides contained the sequence as listed in the figure above (e.g., poly(dA)). Certain wells labelled “control” contained sequences that did not contain the respective sequence, but rather were incubated with nucleoside 5′-triphosphates that were the same nitrogenous base identity. Following immobilization, certain wells labelled “TCEP” were treated with TCEP (100 mM, pH 8.0) for 15 min at 65° C. Following TCEP treatment, all wells were annealed (85° C. for 2 min then 4° C. for 10 min) with their respective complementary strand labelled with a 5′-Cy3 fluorophore. After extensive washing, the strips were analyzed by a fluorescence plate reader (Fluoroskan Ascent) at an excitation wavelength of 534 nm and an emission wavelength of 590 nm.

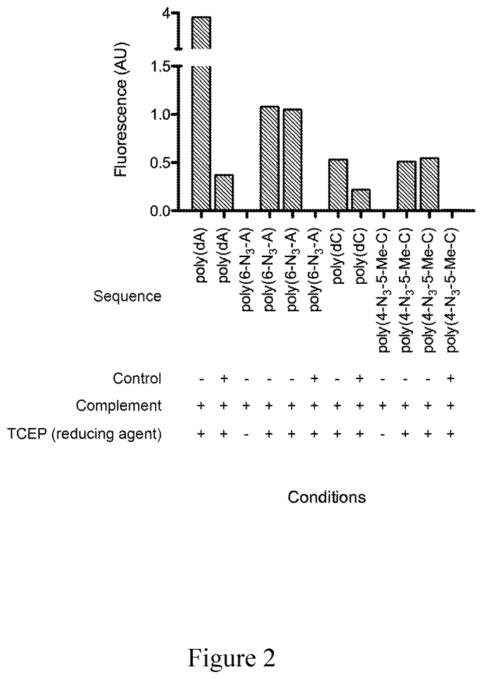

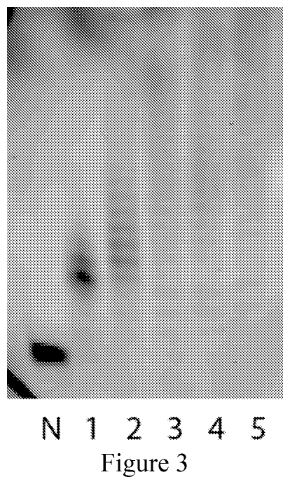

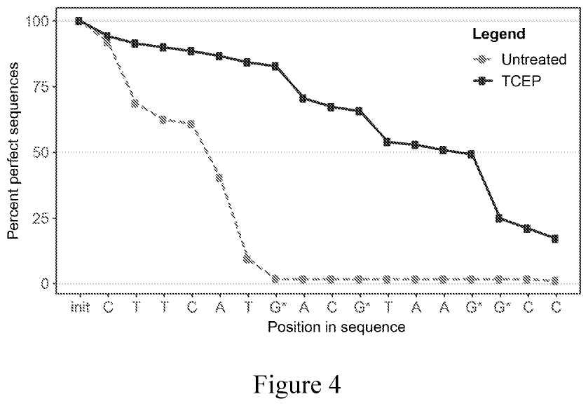

It is evident in FIG. 2 above that amine-masked nucleic acid polymers (e.g., those composed of 6-azido-dA or 4-azido-5-methyl-dC) are unable to hybridize with their respective complementary strand (i.e., poly (dT) or poly (dG), respectively). Controls with canonical poly (dA) and poly (dC) clearly demonstrate that the experimental setup is capable of detecting nucleic acid hybridization, and thus duplex formation through Watson-Crick nucleic acid base-pairing. In the experiment above, when incubated with TCEP, the azide-labelled bases are reduced to the canonical amino bases (e.g., 4-azido-5-methyl-dC to 5-methyl-dC). Upon conversion back to an amino group through TCEP, the amino group is capable of H-bond donation. This conversion from azide to amine is evident as upon incubation of TCEP with nucleic acid polymers containing amine-masked bases, dramatically increase fluorescence is observed, indicating nucleic acid hybridization through Watson-Crick base pairing.

FIG. 3. Engineered TdTs are capable of incorporation of amine-masked nitrogenous base triphosphates to form amine-masked nucleic acid polymers. In this experiment, oligonucleotide homopolymers (poly(dC)) were synthesised in solution. The homopolymers were synthesised using amine-masked 3′-aminoxy 4-azido-5-methyl-2′-deoxycytidine 5′-triphosphate. Nucleotides were first deblocked at the 3′-position with acidic sodium nitrite solution; subsequently, nucleotide tailing with amine-masked methyl-C nucleotides was performed with the 3′-deblocked nucleotides. Nucleotides were present at 0.5 mM in appropriate reaction buffers containing inorganic pyrophosphatase, engineered TdT, and divalent salts. In the gel above, N represents the starting oligonucleotide initiator, 1 is 1 min of reaction, 2 is 5 min of reaction, 3 is 15 min of reaction, 4 is 20 min of reaction, and 5 is 25 min of reaction. Reactions were quenched prior to analysis. Reactions were analysed by PAGE and visualised on a Typhoon scanner by virtue of a covalently attached Cy3 fluorophore.

It is evident in the gel in FIG. 3 that it demonstrates the ability to perform TdT-catalyzed incorporation of 3′-deblocked reversibly terminated dNTPs bearing azido-based amine-masking groups. Nucleic acid polymers containing amine-masked nitrogenous bases prevents (1) nitrogenous base deamination and (2) nucleic acid secondary structure (demonstrated in FIG. 2).

FIG. 4. Effect of non-removal of exocyclic amino blocking group. An oligonucleotide was synthesised by de novo enzymatic nucleic acid synthesis. An engineered terminal deoxynucleotidyl transferase (TdT) was employed to build up the oligonucleotide using reversibly terminated nucleoside triphosphates. A, C, and T nucleotides had canonical bases, i.e. adenine, cytosine, and thymine. The reversibly terminated G nucleotide was base modified to 2-azidoguanine (2-azidoG, shown as G* in the figure). The modified 2-azidoguanine base is unable to hydrogen bond due to the azide moiety masking a key exocyclic amine on the hydrogen bonding face. The synthesised oligonucleotide was analysed by next-generation sequencing (NGS) on an Illumina iSeq 100 with PE 50 reads. Prior to NGS, one portion of the oligonucleotide was treated with 0.1 M tris(2-carboxyethyl)phosphine (TCEP) pH 7.5 at 85° C. for 60 minutes; the other portion was untreated. Both samples were then prepared into an NGS library and amplified by polymerase chain reaction (PCR) with Phire HS DNA polymerase mastermix. The untreated library only yielded 624 reads, all of which terminated before the first 2-azidoguanine addition. In contrast, the TCEP treated library yielded 35,883 reads that reached past 2-azidoguanine positions and included 17% perfect sequences (where all base additions were successful across A, C, 2-azidoG, and T). This figure clearly shows that the presence of 2-azidoG prevents PCR amplification and sequencing of an oligonucleotide. Treatment with TCEP restores 2-azidoG to canonical G and reinstates the ability to amplify by PCR and perform NGS. The masking group thereby is shown to prevent secondary structure in the single stranded nucleic acid.

### DETAILED DESCRIPTION OF THE INVENTION

Disclosed herein are nucleic acid polymers with a portion of the base heterocyclic groups masked with protecting groups to prevent secondary structure formation in the extended strand, thereby improving access of an enzyme to the 3′OH terminus for extension. The polymers may have all of one type of the nitrogenous heterocycles amine masked, or the portion masked may be a mixture of different bases in the polymer. Disclosed herein are nucleic acid polymers with one or more of the amino groups on the base heterocyclic groups masked with protecting groups.

Disclosed is a single stranded nucleic acid polymer, having a portion of at least one type of nitrogenous heterocycle amine masked, wherein the amine masking reduces the formation of secondary structure.

Disclosed is a single stranded nucleic acid polymer with a 3′-O-reversibly terminated 3′-end having a portion of at least one type of nitrogenous heterocycle amine masked, wherein the amine masking reduces the formation of secondary structure.

Therefore, references herein to “3′-blocked”, “3′-reversibly terminated”, or “3′-reversibly terminated nucleotides” refer to nucleic acids which have an additional group at the 3′ position which prevents further addition of nucleotides, i.e., by replacing the 3′—OH group with a protecting group.

It will be understood that references herein to “3′-block”, “3′-blocking group”, “3′-protecting group”, and “3′-reversible terminator” refer to the group attached to the 3′ position of the nucleic acid which prevents further nucleotide addition. The present method uses reversible 3′-blocking groups (3′-reversible terminators) which can be removed by cleavage to allow the addition of further nucleotides. By contrast, irreversible 3′-blocking groups refer to nucleic acids where the 3′—OH group can neither be exposed nor uncovered by cleavage.

The 3′-reversibly terminated nucleoside can be blocked by any chemical group that can be unmasked to reveal a 3′-OH. The 3′-blocked nucleoside triphosphate can be blocked by a 3′ azidomethyl, 3′-aminooxy, 3′-O-allyl group, 3′-O-cyanoethyl, 3′-O-acetyl, 3′-O-nitrate, 3′ phosphate, 3′-O-acetyl levulinic ester, 3′-O-tert butyl dimethyl silane, 3′ trimethyl(silyl)ethoxymethyl, 3′-O-ortho-nitrobenzyl, and 3′-O-para-nitrobenzyl. The 3′-blocked nucleoside triphosphate can be blocked by 3′-O-azidomethyl or 3′-aminooxy.

In one embodiment the single stranded nucleic acid polymer has a 3′ OH end. In one alternative embodiment the single stranded nucleic acid polymer has a 3′O—NH₂ end. In a further alternative embodiment the single stranded nucleic acid polymer has a 3′-O—CH₂N₃ end. In one embodiment of the invention, the amine masked heterocycles may be masked by an azido group, in which case the 3′-end is not 3′-O—CH₂N₃.

One embodiment of the invention is a single stranded nucleic acid polymer having a portion of at least one type of nitrogenous heterocycle amine masked, wherein the amine masked heterocycle is selected from one, two or three of N6-amine masked adenine, N2-amine masked guanine, N4-amine masked cytosine.

One embodiment of the invention is a single stranded nucleic acid polymer having a portion of at least one type of nitrogenous heterocycle amine masked, wherein the amine masked heterocycle is selected from one of N6-amine masked adenine, N2-amine masked guanine, N4-amine masked cytosine.

One embodiment of the invention is a single stranded nucleic acid polymer having a portion of at least one type of nitrogenous heterocycle amine masked, wherein the amine masked heterocycle is selected from two of N6-amine masked adenine, N2-amine masked guanine, N4-amine masked cytosine.

One embodiment of the invention is a single stranded nucleic acid polymer having a portion of at least one type of nitrogenous heterocycle amine masked, wherein the amine masked heterocycle is all three of N6-amine masked adenine, N2-amine masked guanine, N4-amine masked cytosine.

One embodiment of the invention is a single stranded nucleic acid polymer having a portion of at least one type of nitrogenous heterocycle amine masked, wherein the amine masked nitrogenous heterocycles are N2-amine masked guanines.

One embodiment of the invention is a single stranded nucleic acid polymer having a portion of at least one type of nitrogenous heterocycle amine masked, wherein the amine masked nitrogenous heterocycles are N6-amine masked adenines.

One embodiment of the invention is a single stranded nucleic acid polymer having a portion of at least one type of nitrogenous heterocycle amine masked, wherein the amine masked nitrogenous heterocycles are N4-amine masked cytosines.

One embodiment of the invention is a single stranded nucleic acid polymer having a portion of at least one type of nitrogenous heterocycle amine masked, wherein the amine masked nitrogenous heterocycles are N6-amine masked adenine and N2-amine masked guanine.

One embodiment of the invention is a single stranded nucleic acid polymer having a portion of at least one type of nitrogenous heterocycle amine masked wherein the amine masked nitrogenous heterocycles are N6-amine masked adenine and N4-amine masked cytosine.

One embodiment of the invention is a single stranded nucleic acid polymer having a portion of at least one type of nitrogenous heterocycle amine masked, wherein the amine masked nitrogenous heterocycles are N2-amine masked guanine and N4-amine masked cytosine.

In cases where the cytosine bases are not masked, the free NH₂ group may be susceptible to deamination. The deamination turns the C bases to U bases. Strands where the C bases have deaminated can be removed by treatment with a uracil glycosylase which excises the U bases to produce an abasic site. The abasic site can be further digested if required to cleave the strand at the abasic site. Included herein is a purification step of treating the synthesised strands with a uracil glycosylase (UDG). The UDG treatment is particular preferred if the 3′-O blocking moiety is aminooxy, as the nitrite cleavage enhances deamination. The UDG treatment can be performed on the synthesised strand once the final 3′-O blocking group has been removed. The UDG treatment may be performed if the A and/or G bases are masked. Where the C bases are masked, the masking should prevent deamination occurring.

One embodiment of the invention is a method of nucleic acid synthesis comprising:

(a) providing an initiator sequence;

(b) adding extension reagents comprising a 3′-O-reversibly terminated nucleoside triphosphate having an amine-masked nitrogenous heterocycle and a terminal deoxynucleotidyl transferase (TdT) to said initiator sequence to add a single nucleotide to the initiator sequence;

(c) removal of the extension reagents;

(d) cleaving the 3′-O blocking group from the extended nucleic acid polymer;

(e) adding extension reagents comprising a 3′-O-reversibly terminated nucleoside triphosphate having nitrogenous heterocycles with free amine groups and a terminal deoxynucleotidyl transferase (TdT) to said initiator sequence to add a single nucleotide to the initiator sequence;

(f) removal of the extension reagents;

(g) cleaving the 3′-O blocking group from the extended nucleic acid polymer;

(h) repeating steps (b)-(g) to produce an extended nucleic acid polymer having a subset of the amine groups masked amine-masked nitrogenous heterocycles; and

(i) releasing the NH₂ groups on the nitrogenous heterocycles by removing the amine-masks.

The synthesised strands can optionally be treated with a uracil glycosylase in order to remove any deaminated cytosine bases.

One embodiment of the invention is a single stranded nucleic acid polymer comprising formula (I):

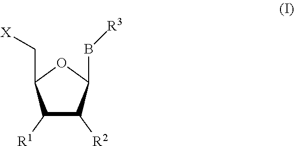

wherein:

R¹ represents O-azidomethyl, aminooxy, O-allyl group, O-cyanoethyl, O-acetyl, O-nitrate, O-phosphate, O-acetyl levulinic ester, O-tert butyl dimethyl silane, O-trimethyl(silyl)ethoxymethyl, O-ortho-nitrobenzyl, or O-para-nitrobenzyl.

R² represents —H, or —OH;

X represents a single stranded nucleic acid polymer having all of at least one type of nitrogenous heterocycle amine masked, wherein the amine masking reduces the formation of secondary structure;

R³ represents an amine masking group; and

B represents a nitrogenous heterocycle.

One embodiment of the invention is a method of nucleic acid synthesis comprising:
(a) providing an initiator sequence;
(b) adding extension reagents comprising a 3′-O—NH₂ blocked or a 3′-O—CH₂N₃ blocked nucleoside triphosphate having an amine-masked nitrogenous heterocycle and a terminal deoxynucleotidyl transferase (TdT) to said initiator sequence to add a single nucleotide to the initiator sequence;
(c) removal of the extension reagents;
(d) cleaving the 3′-O blocking group from the extended nucleic acid polymer;
(e) adding extension reagents comprising a 3′-O—NH₂ blocked or a 3′—O—CH₂N₃ blocked nucleoside triphosphate having nitrogenous heterocycles with free amine groups and a terminal deoxynucleotidyl transferase (TdT) to said initiator sequence to add a single nucleotide to the initiator sequence;
(f) removal of the extension reagents;
(g) cleaving the 3′-O blocking group from the extended nucleic acid polymer;
(h) repeating steps (b)-(g) to produce an extended nucleic acid polymer having a subset of the amine groups masked amine-masked nitrogenous heterocycles; and
(i) releasing the NH₂ groups on the nitrogenous heterocycles by removing the amine-masks.

Disclosed is a single stranded nucleic acid polymer comprising formula (I):

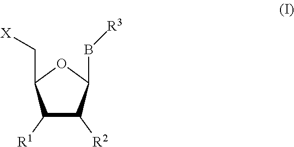

wherein:

R¹ represents —OH, —ONH₂, or —OCH₂N₃;

R² represents —H, or —OH;

X represents a single stranded nucleic acid polymer having a portion of at least one type of nitrogenous heterocycle amine masked, wherein the amine masking reduces the formation of secondary structure;

R³ represents an amine masking group; and

B represents a nitrogenous heterocycle, provided that when R¹ represents —OCH₂N₃, R³ cannot be —N₃.

In one embodiment, formula (I) is selected from:

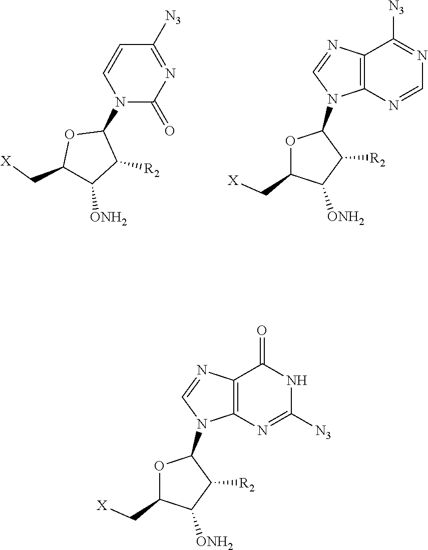

wherein X and R² are as defined herein.

R² may be —OH or —H. In one embodiment, R² is H.

References herein to “amine” refer to a —NH₂ group.

References herein to an “amine masking group” refer to any chemical group which is capable of generating or “unmasking” an amine group which is involved in hydrogen bond base-pairing with a complementary base. Most typically the unmasking will follow a chemical reaction, most suitably a simple, single step chemical reaction. The amine masking group will generally be orthogonal to the 3′-O-blocking group in order to allow selective removal.

Examples of suitable amine masking groups for R³ include azide (—N₃), benzoylamine (N-benzoyl or —NHCOPh), N-methyl (—NHMe), isobutyrylamine, dimethylformamidylamine, 9-fluorenylmethyl carbamate, t-butyl carbamate, benzyl carbamate, acetamide (N-acetyl or —NHCOMe), trifluoroacetamide, pthlamide, benzylamine (N-benzyl or —NH—CH₂-phenyl), triphenylmethylamine, benxylideneamine, tosylamide, isothiocyanate, N-allyl (such as N-dimethylallyl (—NHCH₂—CH═CH₂)) and N-anisoyl (—NHCOPh-OMe), such as azide (—N₃), N-acetyl (—NHCOMe), N-benzyl (—NH—CH₂-phenyl), N-anisoyl (—NHCOPh-OMe), N-methyl, (—NHMe), N-benzoyl (—NHCOPh), N-dimethylallyl (—NHCH₂—CH═CH₂).

In one embodiment, B represents a nitrogenous heterocycle selected from a purine or pyrimidine, or derivative thereof. In a further embodiment, B and R³ can be combined into the following molecular structures, where the nitrogenous heterocycle is connected to the (deoxy)ribose 1′ position of formula (I):

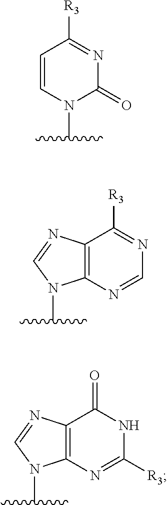

In a further embodiment, R³ represents an azide (—N₃) group and B is selected from:

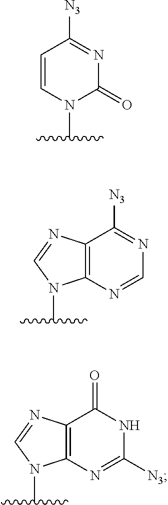 [SMILES: *n1ccc(N=[N+]=[N-])nc1=O ; *n1cnc2c(N=[N+]=[N-])ncnc21 ; *n1cnc2c(=O)[nH]c(N=[N+]=[N-])nc21]

One, two or three of the bases can be N-masked, the other bases being either T/U, having no amine group or being unmasked ‘free’ amines.

This for example some or all of the G bases can be amine masked, and the A bases and C bases can be unmasked. Some or all of the G and C bases can be masked and the A unmasked. Some or all of the G and A bases can be masked and the C unmasked.

The term ‘azide’ or ‘azido’ used herein refers to an —N₃, or more specifically, an —N═N⁺=N⁻ group. It will also be appreciated that azide extends to the presence of a tetrazolyl moiety. The “azide-tetrazole” equilibrium is well known to the skilled person from Lakshman et al (2010) J. Org. Chem. 75, 2461-2473. Thus, references herein to azide extend equally to tetrazole as illustrated below when applied to the R³ groups defined herein:

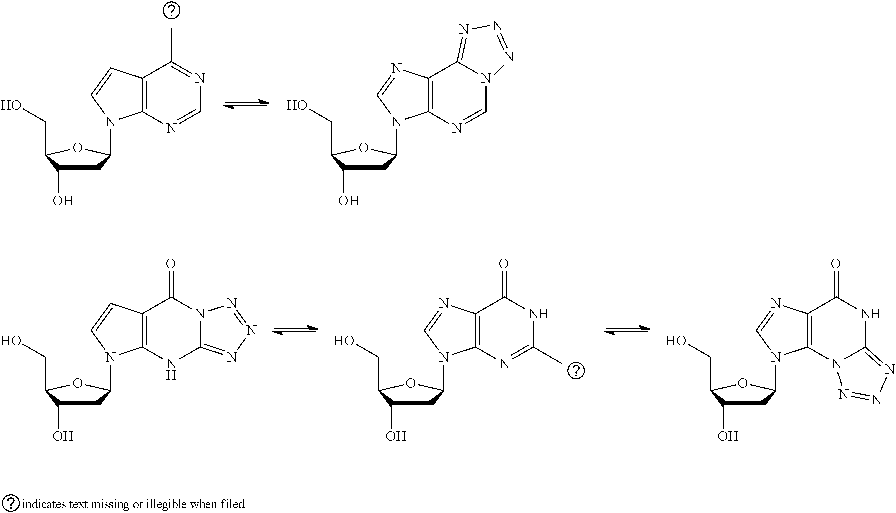

This embodiment has the advantage of reversibly masking the —NH₂ group. While blocked in the —N₃ state, the base (B) is impervious to deamination (e.g., deamination in the presence of sodium nitrite). The base (B) in the N-blocked form is incapable of forming secondary structures via base pairing. Thus even blocking a subset of the free amino groups in the nucleic acid polymer improves the availability of the 3′-end for further extension. The canonical cytosine, adenine, guanine can be respectively recovered from 4-azido cytosine, 6-azido adenine and 2-azido guanine by exposure to a reducing agent (e.g., TCEP). Thus, the —N₃ group serves as an effective protecting group against deamination, especially in the presence of sodium nitrite.

It will be appreciated that the compounds of the invention may be readily applied to methods of enzymatic nucleic acid synthesis which are well known to the person skilled in the art.

Non-limiting methods of nucleic acid synthesis may be found in WO 2016/128731, WO 2016/139477, WO 2017/009663, GB 1613185.6 and GB 1714827.1, the contents of each of which are herein incorporated by reference.

Enzymatic nucleic acid synthesis is defined as any process in which a nucleotide is added to a nucleic acid strand through enzymatic catalysis in the presence or absence of a template.

For example, a method of enzymatic nucleic acid synthesis could include non-templated de novo nucleic acid synthesis utilizing a PoIX family polymerase, such as terminal deoxynucleotidyl transferase, and reversibly terminated 2′-deoxynucleoside 5′-triphosphates or ribonucleoside 5′-triphosphate. Another method of enzymatic nucleic acid synthesis could include templated nucleic acid synthesis, including sequencing-by-synthesis. Reversibly terminated enzymatic nucleic acid synthesis is defined as any process in which a reversibly terminated nucleotide is added to a nucleic acid strand through enzymatic catalysis in the presence or absence of a template. A reversibly terminated nucleotide is a nucleotide containing a chemical moiety that blocks the addition of a subsequent nucleotide. The deprotection or removal of the reversibly terminating chemical moiety on the nucleotide by chemical, electromagnetic, electric current, and/or heat allows the addition of a subsequent nucleotide via enzymatic catalysis. Thus, in one embodiment, the method of enzymatic nucleic acid synthesis is selected from a method of reversibly terminated enzymatic nucleic acid synthesis and a method of templated and non-templated de novo enzymatic nucleic acid synthesis.

The inventors have previously developed a selection of engineered terminal transferase enzymes, any of which may be used to generate the single stranded nucleic acid polymers of the current invention.

Terminal transferase enzymes are ubiquitous in nature and are present in many species. Many known TdT sequences have been reported in the NCBI database www dot ncbi dot nlm dot nih dot gov slash. The sequences of the various described terminal transferases show some regions of highly conserved sequence, and some regions which are highly diverse between different species.

The inventors have modified the terminal transferase from Lepisosteus oculatus TdT (spotted gar) (shown below). However the corresponding modifications can be introduced into the analagous terminal transferase sequences from any other species, including the sequences listed above in the various NCBI entries. The amino acid sequence of the spotted gar (Lepisosteus oculatus) is shown below

wild type spotted Gar TdT
SEQ ID NO: 1:
MLHIPIFPPIKKRQKLPESRNSCKYEVKFSEVAIFLVERKMGSSRRKFLT
NLARSKGFRIEDVLSDAVTHVVAEDNSADELWQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LKTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL

LNRLINRLQNQGILLYYDIVESTFDKTRLPCRKFEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

The inventors have identified various regions in the amino acid sequence having improved properties. Certain regions improve the solubility and handling of the enzyme. Certain other regions improve the ability to incorporate nucleotides with modifications at the 3′-position.

Described herein are modified terminal deoxynucleotidyl transferase (TdT) enzymes comprising amino acid modifications when compared to a wild type sequence SEQ ID NO 1 or a truncated version thereof or the homologous amino acid sequence of a terminal deoxynucleotidyl transferase (TdT) enzyme in other species or the homologous amino acid sequence of Polμ, Polβ, Polλ, and Polθ of any species or the homologous amino acid sequence of X family polymerases of any species, wherein the amino acid is modified at one or more of the amino acids:

V32, A33, 134, F35, A53, V68, V71, E97, 1101, M108, G109, A110, Q115, V116, 5125, T137, Q143, M152, E153, N154, H155, N156, Q157, I158, I165, N169, N173, 5175, E176, G177, P178, C179, L180, A181, F182, M183, R184, A185, L188, H194, A195, I196, S197, S198, S199, K200, E203, G204, D210, Q211, T212, K213, A214, 1216, E217, D218, L220, Y222, V228, D230, Q238, T239, L242, L251, K260, G261, F262, H263, 5264, L265, E267, Q269, A270, D271, N272, A273, H275, F276, T277, K278, M279, Q280, K281, 5291, A292, A293, V294, C295, K296, E298, A299, Q300, A301, Q304, 1305, T309, V310, R311, L312, 1313, A314, 1318, V319, T320, G328, K329, E330, C331, L338, T341, P342, E343, M344, G345, K346, W349, L350, L351, N352, R353, L354, 1355, N356, R357, L358, Q359, N360, Q361, G362, 1363, L364, L365, Y366, Y367, D368, 1369, V370, K376, T377, C381, K383, D388, H389, F390, Q391, K392, F394, 1397, K398, K400, K401, E402, L403, A404, A405, G406, R407, D411, A421, P422, P423, V424, D425, N426, F427, A430, R438, F447, A448, R449, H450, E451, R452, K453, M454, L455, L456, D457, N458, H459, A460, L461, Y462, D463, K464, T465, K466, K467, T474, D477, D485, Y486, 1487, D488, P489.

Modifications which improve the incorporation of modified nucleotides can be at one or more of selected regions shown below. Regions were selected according to mutation data, sequence alignment, and structural data obtained from spotted gar TdT co-crystallized with DNA and a 3′-modified dNTP. The second modification can be selected from one or more of the amino acid regions VAIF, MGA, MENHNQI, SEGPCLAFMRA, HAISSS, DQTKA, KGFHS, QADNA, HFTKMQK, SAAVCK, EAQA, TVRLI, GKEC, TPEMGK, YYDIV, DHFQK, LAAG, APPVDNF, FARHERKMLLDNHALYDKTKK, and DYIDP shown highlighted in bold font in the sequence below.

MLHIPIEPPIKKRQKLPESRNSCKYEVKFSEVAIFLVERKMGSSRRKELT
NLARSKGFRIEDVLSDAVTHVVAEDNSADELWQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LKTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL
LNRLINRLQNQGILLYYDIVESTFDKTRLPCRKEEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

References to particular sequences include truncations thereof. Included herein are modified terminal deoxynucleotidyl transferase (TdT) enzyme comprising at least one amino acid modification when compared to a wild type sequence SEQ ID NO 1 or a truncated version thereof, or the homologous amino acid sequence of a terminal deoxynucleotidyl transferase (TdT) enzyme in other species, wherein the modification is selected from one or more of the amino acid regions WLLNRLINRLQNQGILLYYDIV, VAIF, MGA, MENHNQI, SEGPCLAFMRA, HAISSS, DQTKA, KGFHS, QADNA, HFTKMQK, SAAVCK, EAQA, TVRLI, GKEC, TPEMGK, DHFQK, LAAG, APPVDNF, FARHERKMLLDNHALYDKTKK, and DYIDP of the sequence of SEQ ID NO 1 or the homologous regions in other species.

Truncated proteins may include at least the region shown below (SEQ ID NO 2)

TVSQYACQRRTTMENHNQIETDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHFTKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

Described herein is a modified terminal deoxynucleotidyl transferase (TdT) enzyme comprising at least the sequence:

TVSQYACQRRTTMENHNQIETDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHETKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

or the homologous regions in other species, wherein the sequence has one or more amino acid modifications in one or more of the amino acid regions WLLNRLINRLQNQGILLYYDI, MENHNQI, SEGPCLAFMRA, HAISSS, DQTKA, KGFHS, QADNA, HFTKMQK, SAAVCK, EAQA, TVRLI, GKEC, TPEMGK, DHFQK, LAAG, APPVDNF, FARHERKMLLDNHALYDKTKK, and DYIDP of the sequence.

TVSQYACQRRTTMENHNQIFTDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHFTKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

Sequence homology extends to all modified or wild-type members of family X polymerases, such as DNA Polμ (also known as DNA polymerase mu or POLM), DNA Polβ (also known as DNA polymerase beta or POLB), and DNA Polλ (also known known as DNA polymerase lambda or POLL). It is well known in the art that all family X member polymerases, of which TdT is a member, either have terminal transferase activity or can be engineered to gain terminal transferase activity akin to terminal deoxynucleotidyl transferase (Biochim Biophys Acta. 2010 May; 1804(5): 1136-1150). For example, when the following human TdT loop1 amino acid sequence

...ESTFEKLRLPSRKVDALDHF...

was engineered to replace the following human Polμ amino acid residues

...HSCCESPTRLAQQSHMDAF...,

the chimeric human Polμ containing human TdT loop1 gained robust terminal transferase activity (Nucleic Acids Res. 2006 September; 34(16): 4572-4582).

Furthermore, it was generally demonstrated in US patent application no. 2019/0078065 that family X polymerases when engineered to contain TdT loop1 chimeras could gain robust terminal transferase activity. Additionally, it was demonstrated that TdT could be converted into a template-dependent polymerase through specific mutations in the loop1 motif (Nucleic Acids Research, June 2009, 37(14):4642-4656). As it has been shown in the art, family X polymerases can be trivially modified to either display template-dependent or template-independent nucleotidyl transferase activities. Therefore, all motifs, regions, and mutations demonstrated in this patent can be trivially extended to modified X family polymerases to enable modified X family polymerases to incorporate 3′-modified nucleotides, reversibly terminated nucleotides, and modified nucleotides in general to effect methods of nucleic acid synthesis.

Modifications which improve the solubility include a modification within the amino acid region WLLNRLINRLQNQGILLYYDIV shown highlighted in bold font in the sequence below.

MLHIPIFPPIKKRQKLPESRNSCKYEVKFSEVAIFLVERKMGSSRRKFLT
NLARSKGFRIEDVLSDAVTHVVAEDNSADELWQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LKTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL
LNRLINRLQNQGILLYYDIVESTFDKTRLPCRKFEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

Modifications which improve the incorporation of modified nucleotides can be at one or more of selected regions shown below. The second modification can be selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP shown highlighted in bold font in the sequence below.

MLHIPIFPPIKKRQKLPESRNSCKYEVKFSEVAIFLVERKMGSSRRKFLT
NLARSKGFRIEDVLSDAVTHVVAEDNSADELWQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LKTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL
LNRLINRLQNQGILLYYDIVESTFDKTRLPCRKFEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

Described herein is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid regions WLLNRLINRLQNQGILLYYDI, VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

Homologous refers to protein sequences between two or more proteins that possess a common evolutionary origin, including proteins from superfamilies in the same species of organism as well as homologous proteins from different species. Such proteins (and their encoding nucleic acids) have sequence homology, as reflected by their sequence similarity, whether in terms of percent identity or by the presence of specific residues or motifs and conserved positions. A variety of protein (and their encoding nucleic acid) sequence alignment tools may be used to determine sequence homology. For example, the Clustal Omega multiple sequence alignment program provided by the European Molecular Biology Laboratory (EMBL) can be used to determine sequence homology or homologous regions.

Improved sequences as described herein can contain both modifications, namely

a. a first modification is within the amino acid region WLLNRLINRLQNQGILLYYDI of the sequence of SEQ ID NO 1 or the homologous region in other species; and

b. a second modification is selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

Disclosed is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid regions WLLNRLINRLQNQGILLYYDI, VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

Further disclosed is a modified terminal deoxynucleotidyl transferase (TdT) enzyme comprising at least two amino acid modifications when compared to a wild type sequence SEQ ID NO 1 or the homologous amino acid sequence of a terminal deoxynucleotidyl transferase (TdT) enzyme in other species, wherein;

a. a first modification is within the amino acid region WLLNRLINRLQNQGILLYYDIV of the sequence of SEQ ID NO 1 or the homologous region in other species; and

b. a second modification is selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

For the purposes of brevity, the modifications are further described in relation to SEQ ID NO 1, but the modifications are applicable to the sequences from other species, for example those sequences listed above having sequences in the NCBI database.

The modification within the region WLLNRLINRLQNQGILLYYDIV or the corresponding region from other species help improve the solubility of the enzyme. The modification within the amino acid region WLLNRLINRLQNQGILLYYDIV can be at one or more of the underlined amino acids.

Particular changes can be selected from W-Q, N-P, R-K, L-V, R-L, L-W, Q-E, N-K, Q-K or I-L.

The sequence WLLNRLINRLQNQGILLYYDIV can be altered to QLLPKVINLWEKKGLLLYYDLV.

The second modification improves incorporation of nucleotides having a modification at the 3′ position in comparison to the wild type sequence. The second modification can be selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species. The second modification can be selected from two or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species shown highlighted in bold font in the sequence below.

SEQ ID NO: 1:
MLHIPIFPPIKKRQKLPESRNSCKYEVKFSEVAIFLVERKMGSSRRKFLT
NLARSKGFRIEDVLSDAVTHVVAEDNSADELWQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LKTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL
LNRLINRLQNQGILLYYDIVESTFDKTRLPCRKFEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

The identified positions commence at positions V32, E74, M108, F182, T212, D271, M279, E298, A421, L456, Y486. Modifications disclosed herein contain at least one modification at the defined positions.

The modified amino acid can be in the region FMRA. The modified amino acid can be in the region QADNA. The modified amino acid can be in the region EAQA. The modified amino acid can be in the region APP. The modified amino acid can be in the region LDNHA. The modified amino acid can be in the region YIDP. The region FARHERKMLLDNHA is advantageous for removing substrate biases in modifications. The FARHERKMLLDNHA region appears highly conserved across species.

The modification selected from one or more of the amino acid regions FMRA, QADNA, EAQA, APP, FARHERKMLLDNHA, and YIDP can be at the underlined amino acid(s).

The positions for modification can include A53, V68, V71, D75, E97, 1101, G109, Q115, V116, S125, T137, Q143, N154, H155, Q157, I158, I165, G177, L180, A181, M183, A195, K200, T212, K213, A214, E217, T239, F262, 5264, Q269, N272, A273, K281, 5291, K296, Q300, T309, R311, E330, T341, E343, G345, N352, N360, Q361, 1363, Y367, H389, L403, G406, D411, A421, P422, V424, N426, R438, F447, R452, L455, and/or D488.

Amino acid changes include any one of A53G, V681, V711, D75N, D75Q, E97A, 1101V, G109E, G109R, Q115E, V1161, V116S, S125R, T137A, Q143P, N154H, H155C, Q157K, Q157R, 1158M, I165V, G177D, L180V, A181E, M183R, A195P, K200R, T212S, K213S, A214R, E217Q, T239S, F262L, 5264T, Q269K, N272K, A273S, A273T, K281R, 5291N, K296R, Q300D, T309A, R311W, E330N, T341S, E343Q, G345R, N352Q, N360K, Q361K, 1363L, Y367C, H389A, L403R, G406R, D411N, A421L, A421M, A421V, P422A, P422C, V424Y, N426R, R438K, F447W, R452K, L4551, and/or D488P.

Amino acid changes include any two or more of A53G, V681, V711, D75N, D75Q, E97A, 1101V, G109E, G109R, Q115E, V1161, V116S, S125R, T137A, Q143P, N154H, H155C, Q157K, Q157R, I158M, I165V, G177D, L180V, A181E, M183R, A195P, K200R, T212S, K213S, A214R, E217Q, T239S, F262L, S264T, Q269K, N272K, A273S, A273T, K281R, S291N, K296R, Q300D, T309A, R311W, E330N, T341S, E343Q, G345R, N352Q, N360K, Q361K, 1363L, Y367C, H389A, L403R, G406R, D411N, A421L, A421M, A421V, P422A, P422C, V424Y, N426R, R438K, F447W, R452K, L4551, and/or D488P.

The modification of QADNA to KADKA, QADKA, KADNA, QADNS, KADNT, or QADNT is advantageous for the incorporation of 3′-O-modified nucleoside triphosphates to the 3′-end of nucleic acids and removing substrate biases during the incorporation of modified nucleoside triphosphates. The modification of APPVDN to MCPVDN, MPPVDN, ACPVDR, VPPVDN, LPPVDR, ACPYDN, LCPVDN, or MAPVDN is advantageous for the incorporation of 3′-O-modified nucleoside triphosphates to the 3′-end of nucleic acids and removing substrate biases during the incorporation of modified nucleoside triphosphates. The modification of FARHERKMLLDRHA to WARHERKMILDNHA, FARHERKMILDNHA, WARHERKMLLDNHA, FARHERKMLLDRHA, or FARHEKKMLLDNHA is also advantageous for the incorporation of 3′-O-modified nucleoside triphosphates to the 3′-end of nucleic acids and removing substrate biases during the incorporation of modified nucleoside triphosphates.

The modification can be selected from one or more of the following sequences FRRA, QADKA, EADA, MPP, FARHERKMLLDRHA, and YIPP. Included is a modified terminal deoxynucleotidyl transferase (TdT) enzyme wherein the second modification is selected from two or more of the following sequences FRRA, QADKA, EADA, MPP, FARHERKMLLDRHA, and YIPP. Included is a modified terminal deoxynucleotidyl transferase (TdT) enzyme wherein the second modification contains each of the following sequences FRRA, QADKA, EADA, MPP, FARHERKMLLDRHA, and YIPP.

In order to aid purification of the expressed sequence, the amino acid can be further modified. For example the amino acid sequence can contain one or more further histidine residues at the terminus.

References herein to a deoxyribo derivative of adenosine, guanosine and cytidine refer to deoxy derivatives thereof (i.e. deoxyadenosine, deoxyguanosine and deoxycytidine) and the phosphated derivatives thereof (i.e. adenosine monophosphate, adenosine diphosphate, adenosine triphosphate, guanosine monophosphate, guanosine diphosphate, guanosine triphosphate, cytidine monophosphate, cytidine diphosphate, cytidine triphosphate and all the deoxyribose versions thereof).

References herein to ‘nucleoside triphosphates’ refer to a molecule containing a nucleoside (i.e. a base attached to a deoxyribose or ribose sugar molecule) bound to three phosphate groups. Examples of nucleoside triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleoside triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleoside triphosphates, such as naturally occurring modified nucleosides and artificial nucleosides.

Therefore, references herein to ‘3’-blocked nucleoside triphosphates' refer to nucleoside triphosphates (e.g., dATP, dGTP, dCTP or dTTP) which have an additional group on the 3′ end which prevents further addition of nucleotides, i.e., by replacing the 3′—OH group with a protecting group.

It will be understood that references herein to ‘3′-block’, ‘3′-blocking group’ or ‘3′-protecting group’ refer to the group attached to the 3′ end of the nucleic acid or nucleoside triphosphate which prevents further nucleotide addition. The present method uses reversible 3′-blocking groups which can be removed by cleavage to allow the addition of further nucleotides. By contrast, irreversible 3′-blocking groups refer to dNTPs where the 3′—OH group can neither be exposed nor uncovered by cleavage. The 3′-blocked nucleoside triphosphate can be blocked by a 3′-O-azidomethyl or 3′-aminooxy.

The blocking group on the 3′-end should be orthogonal to the group masking the amine group on the base so as the groups can be separately removed.

References herein to ‘cleaving agent’ refer to a substance which is able to cleave the 3′-blocking group from the 3′-blocked nucleoside triphosphate. In one embodiment, the cleaving agent is a chemical cleaving agent.

It will be understood by the person skilled in the art that the selection of cleaving agent is dependent on the type of 3′-nucleotide blocking group used. For instance, tris(2-carboxyethyl)phosphine (TCEP) or tris(hydroxypropyl)phosphine (THPP) can be used to cleave a 3′-O-azidomethyl group, or sodium nitrite can be used to cleave a 3′-aminoxy group. Therefore, in one embodiment, the cleaving agent is selected from: tris(2-carboxyethyl)phosphine (TCEP) or sodium nitrite.

In one embodiment, the cleaving agent is added in the presence of a cleavage solution comprising a denaturant, such as urea, guanidinium chloride, formamide or betaine. The addition of a denaturant has the advantage of being able to further disrupt any undesirable secondary structures in the single stranded DNA. In a further embodiment, the cleavage solution comprises one or more buffers. It will be understood by the person skilled in the art that the choice of buffer is dependent on the exact cleavage chemistry and cleaving agent required.

References herein to an ‘initiator sequence’ refer to a short oligonucleotide with a free 3′-end which the 3′-blocked nucleoside triphosphate can be attached to. In one embodiment, the initiator sequence is a DNA initiator sequence. In an alternative embodiment, the initiator sequence is an RNA initiator sequence.

References herein to a ‘DNA initiator sequence’ refer to a small sequence of DNA which the 3′-blocked nucleoside triphosphate can be attached to, i.e., DNA will be synthesised from the end of the DNA initiator sequence. In one embodiment, the initiator sequence is between 5 and 50 nucleotides long, such as between 5 and 30 nucleotides long (i.e. between 10 and 30), in particular between 5 and 20 nucleotides long (i.e., approximately 20 nucleotides long), more particularly 5 to 15 nucleotides long, for example 10 to 15 nucleotides long, especially 12 nucleotides long.

In one embodiment, the initiator sequence is single-stranded. In an alternative embodiment, the initiator sequence is double-stranded. It will be understood by persons skilled in the art that a 3′-overhang (I.e., a free 3′-end) allows for efficient addition.

In one embodiment, the initiator sequence is immobilised on a solid support. This allows TdT and the cleaving agent to be removed without washing away the synthesised nucleic acid. The initiator sequence may be attached to a solid support stable under aqueous conditions so that the method can be easily performed via a flow setup.

In one embodiment, the initiator sequence is immobilised on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In one embodiment, the initiator sequence contains a base or base sequence recognisable by an enzyme. A base recognised by an enzyme, such as a glycosylase, may be removed to generate an abasic site which may be cleaved by chemical or enzymatic means. A base sequence may be recognised and cleaved by a restriction enzyme.

In a further embodiment, the initiator sequence is immobilised on a solid support via an orthogonal chemically-cleavable linker, such as a disulfide, allyl, or azide-masked hemiaminal ether linker. Therefore, in one embodiment, where neither the N-masking group or the 3′-block are azido, the method additionally comprises extracting the resultant nucleic acid by cleaving the chemical linker through the addition of tris(2-carboxyethyl)phosphine (TCEP) or dithiothreitol (DTT) for a disulfide linker; palladium complexes or an allyl linker; or TCEP for an azide-masked hemiaminal ether linker.

In one embodiment, the resultant nucleic acid is extracted and amplified by polymerase chain reaction using the nucleic acid bound to the solid support as a template. The initiator sequence could therefore contain an appropriate forward primer sequence and an appropriate reverse primer could be synthesised.

In one embodiment, the terminal deoxynucleotidyl transferase (TdT) of the invention is added in the presence of an extension solution comprising one or more buffers (e.g., Tris or cacodylate), one or more salts (e.g., Na⁺, K⁺, Mg²⁺, Mn²⁺, Cu²⁺, Zn²⁺, Co²⁺, etc. all with appropriate counterions, such as CI) and inorganic pyrophosphatase (e.g., the Saccharomyces cerevisiae homolog). It will be understood that the choice of buffers and salts depends on the optimal enzyme activity and stability. The use of an inorganic pyrophosphatase helps to reduce the build-up of pyrophosphate due to nucleoside triphosphate hydrolysis by TdT. Therefore, the use of an inorganic pyrophosphatase has the advantage of reducing the rate of (1) backwards reaction and (2) TdT strand dismutation.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

In one embodiment, steps (d) and (g) are performed at a temperature less than 99° C., such as less than 95° C., 90° C., 85° C., 80° C., 75° C., 70° C., 65° C., 60° C., 55° C., 50° C., 45° C., 40° C., 35° C., or 30° C. It will be understood that the optimal temperature will depend on the cleavage agent utilised. The temperature used helps to assist cleavage and disrupt any secondary structures formed during nucleotide addition.

In one embodiment, steps (c) and (f) are performed by applying a wash solution. In one embodiment, the wash solution comprises the same buffers and salts as used in the extension solution described herein. This has the advantage of allowing the wash solution to be collected after step (c) and recycled as extension solution in step (b) when the method steps are repeated.

### EXAMPLES

The following studies illustrate the invention:

### Example 1: Engineered TdTs are Capable of Incorporation of Amine-Masked Nitrogenous Base Trisphosphates to Form Amine-Masked Nucleic Acid Polymers

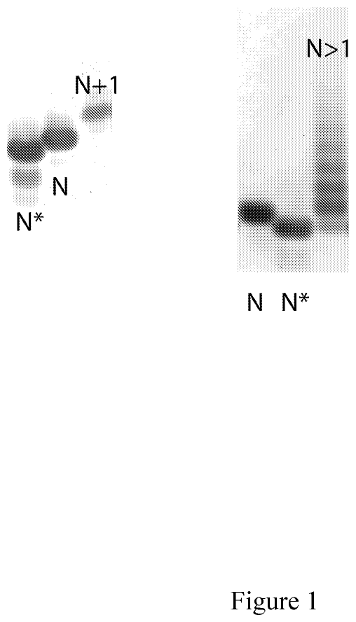

Shown in FIG. 1 are examples of TdT-catalyzed addition of amine-masked nucleoside triphosphates to form amine masked nucleic acid polymers. In the left hand example, the amine-masked 4-azido-5-methyl-2′-deoxy-3′-aminoxy-cytidine 5′-triphosphate is added to a nucleic acid of length N (see left gel, N versus N+1) by TdT. N* is a 3′-phosphorylated version of N. In the right hand example, 6-azido-2′-deoxyadenosine 5′-triphosphate is added to a nucleic acid of length N (see right gel, N versus N+1) by TdT. By virtue of the unblocked 3′-hydroxyl, a tail of 6-azido-2′-deoxyadenosine is made to form an amine-masked nucleic acid polymer. All reactions were run in an appropriate buffer containing the nucleoside 5′-triphosphate (0.5 mM), inorganic pyrophosphatase, engineered TdT, and divalent salts. Reactions were analyzed by denaturing PAGE and visualized by SybrGold staining.

This example demonstrates that TdT is capable of performing both controlled and uncontrolled enzymatic DNA synthesis utilizing amine-masked nucleotides.

### Example 2: Amine-Masked Nucleic Acid Polymers do not Support Secondary Structure Duplex Formation

In this experiment, 5′-biotynlated oligonucleotide homopolymers were immobilized on a neutravidin plate (High Binding Capacity 96-well Strips, Thermo Fisher Scientific). These oligonucleotides contained the sequence as listed in the figure above (e.g., poly(dA)). Certain wells labelled “control” contained sequences that did not contain the respective sequence, but rather were incubated with nucleoside 5′-triphosphates that were the same nitrogenous base identity. Following immobilization, certain wells labelled “TCEP” were treated with TCEP (100 mM, pH 8.0) for 15 min at 65° C. Following TCEP treatment, all wells were annealed (85° C. for 2 min then 4° C. for 10 min) with their respective complementary strand labelled with a 5′-Cy3 fluorophore. After extensive washing, the strips were analyzed by a fluorescence plate reader (Fluoroskan Ascent) at an excitation wavelength of 534 nm and an emission wavelength of 590 nm.

It is evident in FIG. 2 above that amine-masked nucleic acid polymers (e.g., those composed of 6-azido-dA or 4-azido-5-methyl-dC) are unable to hybridize with their respective complementary strand (i.e., poly(dT) or poly(dG), respectively). Controls with canonical poly (dA) and poly(dC) clearly demonstrate that the experimental setup is capable of detecting nucleic acid hybridization, and thus duplex formation through Watson-Crick nucleic acid base-pairing. In the experiment above, when incubated with TCEP, the azide-labelled bases are reduced to the canonical amino bases (e.g., 4-azido-5-methyl-dC to 5-methyl-dC). Upon conversion back to an amino group through TCEP, the amino group is capable of H-bond donation. This conversion from azide to amine is evident as upon incubation of TCEP with nucleic acid polymers containing amine-masked bases, dramatically increase fluorescence is observed, indicating nucleic acid hybridization through Watson-Crick base pairing.

### Example 3: Engineered TdTs are Capable of Incorporation of Amine-Masked Nitrogenous Base Triphosphates to Form Amine-Masked Nucleic Acid Polymers

In this experiment, oligonucleotide homopolymers (poly(dC)) were synthesised in solution. The homopolymers were synthesised using amine-masked 3′-aminoxy 4-azido-5-methyl-2′-deoxycytidine 5′-triphosphate. Nucleotides were first deblocked at the 3′-position with acidic sodium nitrite solution; subsequently, nucleotide tailing with amine-masked methyl-C nucleotides was performed with the 3′-deblocked nucleotides. Nucleotides were present at 0.5 mM in appropriate reaction buffers containing inorganic pyrophosphatase, engineered TdT, and divalent salts. In the gel above, N represents the starting oligonucleotide initiator, 1 is 1 min of reaction, 2 is 5 min of reaction, 3 is 15 min of reaction, 4 is 20 min of reaction, and 5 is 25 min of reaction. Reactions were quenched prior to analysis. Reactions were analysed by PAGE and visualised on a Typhoon scanner by virtue of a covalently attached Cy3 fluorophore.

It is evident in the gel in FIG. 3 that it demonstrates the ability to perform TdT-catalyzed incorporation of 3′-deblocked reversibly terminated dNTPs bearing azido-based amine-masking groups. Nucleic acid polymers containing amine-masked nitrogenous bases prevents (1) nitrogenous base deamination and (2) nucleic acid secondary structure (demonstrated in FIG. 2).

### Example 4: Synthesis of 4-azido-5-methyl-2′-deoxy-3′-aminoxy-cytidine 5′-triphosphate

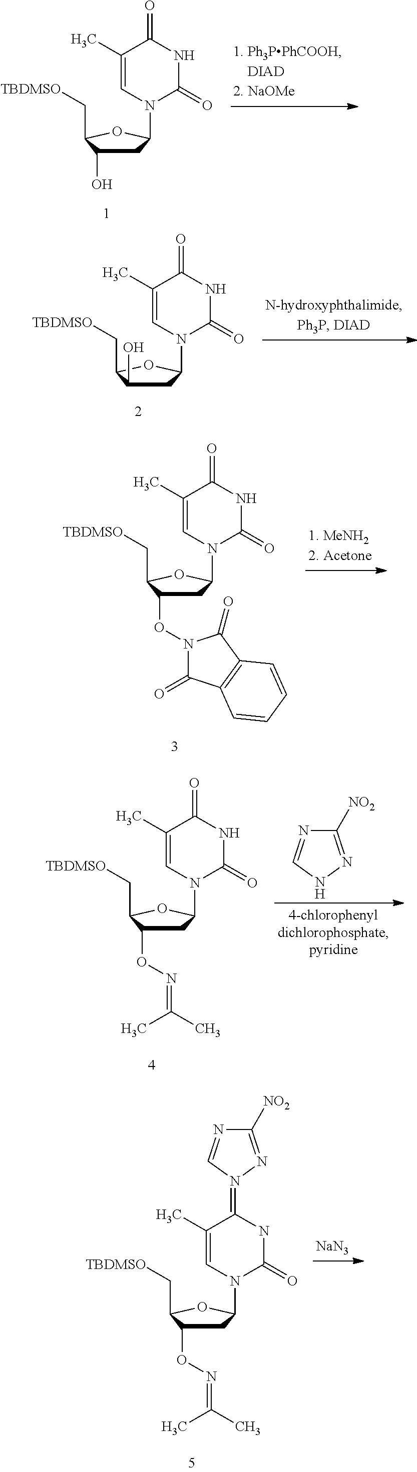

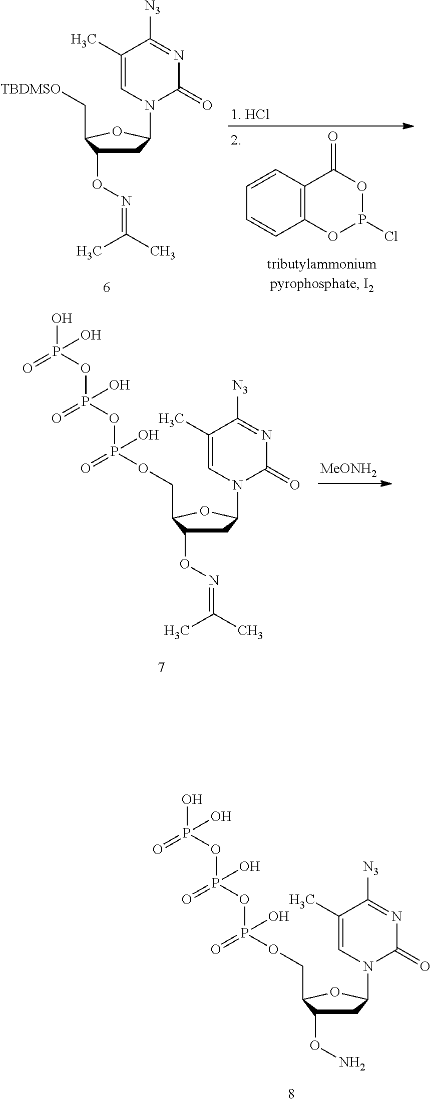

Starting with 5′-TBDMS thymidine 1 (Ogilvie, K. K. Can. J. Chem., 1973, 51, 3799-3807), the 3′-stereocentre was inverted by Mitsunobu reaction with benzoic acid followed by methanolysis with sodium hydroxide in methanol to give alcohol 2. The 3′-aminoxy substituent was introduced by Mitsunobu reaction with N-hydroxyphthalimide followed by cleavage of the phthalimide group of compound 3 with methylamine and protection of the aminoxy group as the acetone oxime 4. The 4-azide substituent was introduced by activation of the 4-carbonyl group of compound to the 3-nitro-1,2,4-triazolide 5 with 4-chlorophenyl dichlorophosphate and 3-nitro-1,2,4-triazole in pyridine, followed by reaction with sodium azide to provide azide 6. After cleavage of the 5′-TBDMS protecting group, the triphosphate group was introduced using the Ludwig-Eckstein method (Ludwig, J.; Eckstein, F., J. Org. Chem., 1989, 54, 631-635). Finally, the acetone oxime protecting group of compound 7 was cleaved with aqueous methoxylamine to provide the 3′-aminoxy triphosphate 8.

### Example 5: Synthesis of 6-azido-2′-deoxy-adenosine 5′-triphosphate

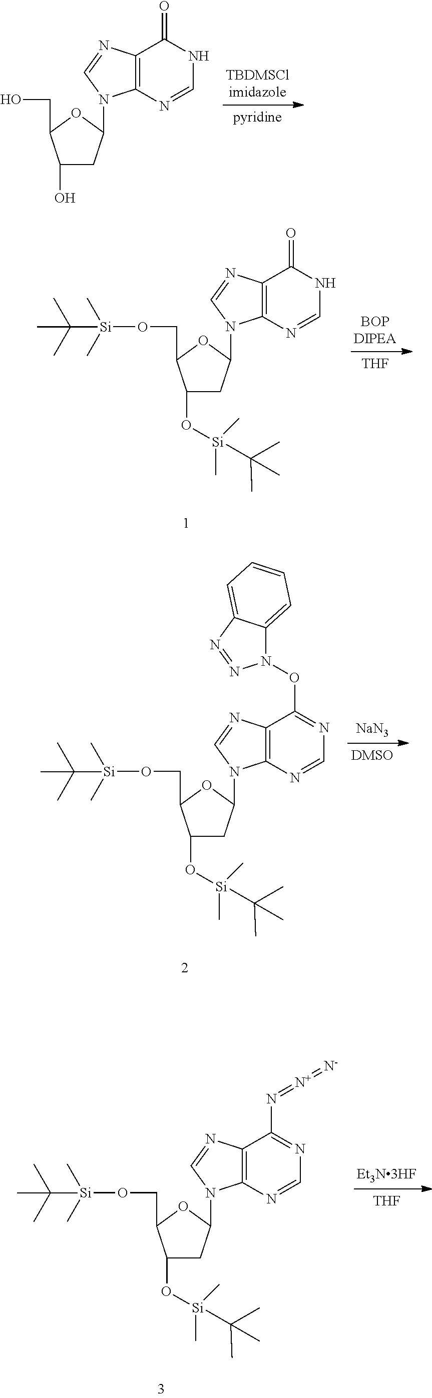

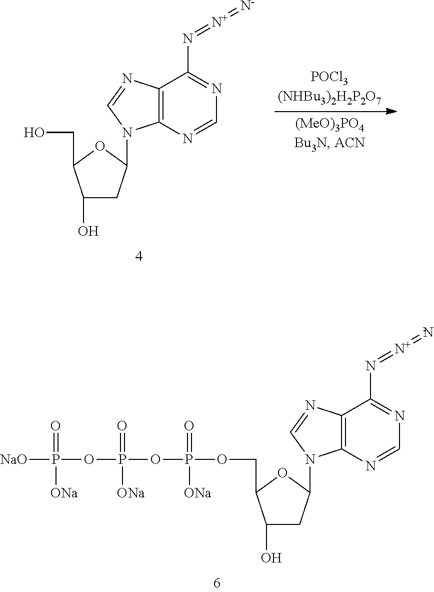

The TBDMS protected 2-deoxyinosine 1 [Can. J. Chem. 1973, 51, 3799-3807] was subjected to activation of C-6 amide carbonyl with 1H-benzotriazol-1-yloxy-tris(dimethylamino) phosphonium hexafluorophosphate (BOP) in the presence of N,N-diisopropylethylamine (DIPEA) [J. Org. Chem. 2010, 75, 2461-2473] to provide O 6-(benzotriazol-1-yl) derivatives 2. Next, displacement reactions by sodium azide followed by removal of silicon-based protection of compound 3 with triethylamine trihydrofluoride [Bioorg. Med. Chem. Lett. 1944, 11(4), 1345-1346] gave nucleoside 4. Finally, the unprotected nucleoside 4 was phosphorylated according to the Ludwig-Eckstein procedure to yield triphosphate 5 (J. Org. Chem., 1989, 54, 631-635).

### Example 6: Synthesis of 6-azido-2′-deoxy-3′-aminoxy-adenosine 5′-triphosphate

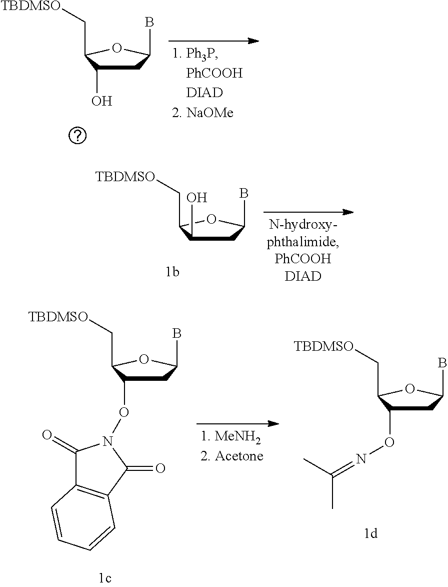

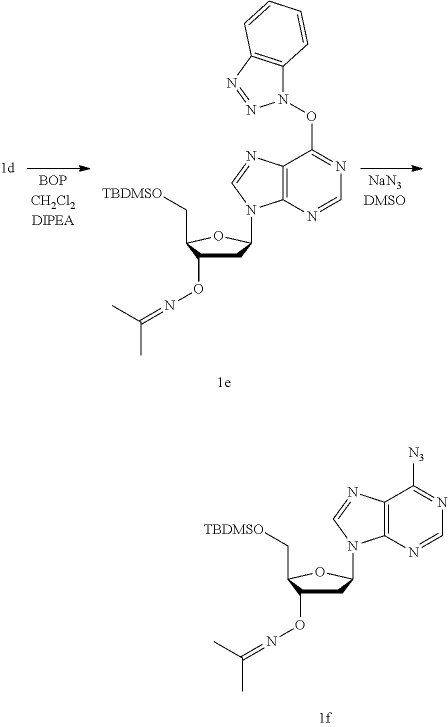

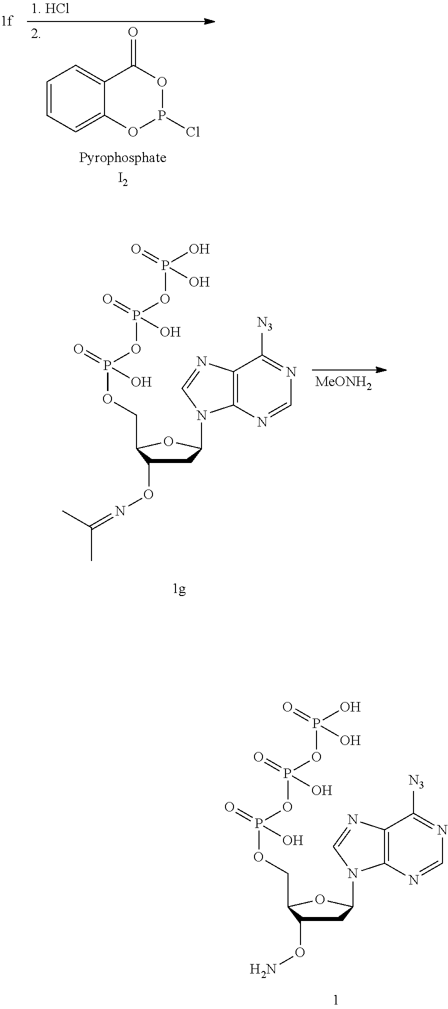

Starting with 5′-TBDMS deoxyinosine 1, the 3′-stereocentre was inverted by Mitsunobu reaction with benzoic acid followed by methanolysis with sodium hydroxide in methanol to give alcohol 1b. The 3′-aminoxy substituent was introduced by Mitsunobu reaction with N-hydroxyphthalimide followed by cleavage of the phthalimide group of compound 1c with methylamine and protection of the aminoxy group as the acetone oxime 1d. The 6-azide substituent was introduced by activation of the 6-carbonyl group with benzotriazol-1-yloxytris(dimethylamino)phosphonium to yield 1e, followed by reaction with sodium azide to provide azide 1f. After cleavage of the 5′-TBDMS protecting group, the triphosphate group was introduced using the Ludwig-Eckstein method (Ludwig, J.; Eckstein, F., J. Org. Chem., 1989, 54, 631-635). Finally, the acetone oxime protecting group of compound 7 was cleaved with aqueous methoxylamine to provide the 3′-aminoxy triphosphate 8.

### Example 7: Enzymatic DNA Synthesis Using Azide-Masked Nitrogenous Heterocycles

In the following method of DNA synthesis, engineered terminal deoxynucleotidyl transferase is used to add 3′-O-aminoxy reversibly terminated 2′-deoxynucleoside 5′-triphosphates to the 3′-end of DNA strands. This addition process is repeated until a desired sequence is synthesized.

The 3′-O-aminoxy moiety must be deaminated (e.g., with acidic sodium nitrite) after each addition cycle to effect reversible termination. The process of deamination after each addition cycle also results in the mutagenic deamination of nitrogenous heterocycles containing amines (e.g., adenine, cytosine and guanine).

Thus, in this example, amino moieties on the nitrogenous heterocycles are masked with an azido group to prevent secondary structure formation. For example, one or a combination of 2′-deoxy-3′-O-aminoxy-N4-azidocytidine 5′-triphosphate, 2′-deoxy-3′-O-aminoxy-N6-azidoadenine 5′-triphosphate, 2′-deoxy-3′-O-aminoxy-N2-azidoguanosine 5′-triphosphate and 2′-deoxy-3′-O-aminoxy-5-ethyluridine 5′-triphosphate are used as nucleotide building blocks during each addition cycle in the presence of engineered TdT and required buffer components.

A DNA polymer with amine-masked nitrogenous heterocycles (e.g., N4-azidocytosine, N6-azidoadenine, N2-azidoguanine) is thus synthesized. All amine-masked nitrogenous heterocycles are unmasked to reveal an amino group through exposure to a reducing agent (e.g., TCEP). The DNA polymer is now composed of nitrogenous heterocycles with unmasked amino groups (e.g., N4-azidocytosine is unmasked to cytosine, N6-azidoadenine is unmasked to adenine and N2-azidoguanine is unmasked to guanine). The DNA polymer can now be used for downstream molecular biology applications.

1. A single stranded nucleic acid polymer with a 3′-O-reversibly terminated 3′-end having a portion of at least one type of nitrogenous heterocycle amine masked, wherein the amine masking reduces the formation of secondary structure.

2. A single stranded nucleic acid polymer according to claim 1 with a 3′ O—NH₂ end having a portion of the nitrogenous heterocycles amine masked, wherein the amine masking reduces the formation of secondary structure.

3. A single stranded nucleic acid polymer according to claim 1 with a 3′O—CH₂N₃ end having a portion of the nitrogenous heterocycles amine masked, wherein the amine masking reduces the formation of secondary structure.

4. A single stranded polymer according to claim 1 having all of at least one type of nitrogenous heterocycle amine masked.

5. The nucleic acid polymer according claim 1, wherein the amine masked heterocycle is selected from one, two or three of N6-amine masked adenine, N2-amine masked guanine, N4-amine masked cytosine.

6. The nucleic acid polymer according to claim 5, wherein said amine masked heterocycles are masked by an azido group.

7. The nucleic acid polymer according to claim 5, wherein the amine masked heterocycle is selected from one of N6-amine masked adenine, N2-amine masked guanine, N4-amine masked cytosine.

8. The nucleic acid polymer according to claim 5, wherein the amine masked heterocycle is selected from two of N6-amine masked adenine, N2-amine masked guanine, N4-amine masked cytosine.

9. The nucleic acid polymer according to claim 5, wherein the amine masked heterocycle is all three of N6-amine masked adenine, N2-amine masked guanine, N4-amine masked cytosine.

10. The nucleic acid polymer according to claim 7, wherein the amine masked nitrogenous heterocycles are N2-amine masked guanines.

11. The nucleic acid polymer according to claim 7, wherein the amine masked nitrogenous heterocycles are N6-amine masked adenines.

12. The nucleic acid polymer according to claim 7, wherein the amine masked nitrogenous heterocycles are N4-amine masked cytosines.

13. The nucleic acid polymer according to claim 8, wherein the amine masked nitrogenous heterocycles are N6-amine masked adenine and N2-amine masked guanine.

14. The nucleic acid polymer according to claim 8, wherein the amine masked nitrogenous heterocycles are N6-amine masked adenine and N4-amine masked cytosine.

15. The nucleic acid polymer according to claim 8, wherein the amine masked nitrogenous heterocycles are N2-amine masked guanine and N4-amine masked cytosine.

16. The single stranded nucleic acid polymer according to claim 1 comprising formula (I): 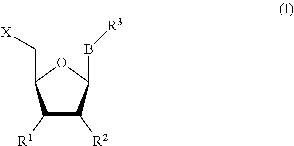
wherein:
R¹ represents O-azidomethyl, aminooxy, O-allyl group, O-cyanoethyl, O-acetyl, O-nitrate, O-phosphate, O-acetyl levulinic ester, O-tert butyl dimethyl silane, O-trimethyl(silyl)ethoxymethyl, O-ortho-nitrobenzyl, or O-para-nitrobenzyl.
R² represents —H, or —OH;
X represents a single stranded nucleic acid polymer having all of at least one type of nitrogenous heterocycle amine masked, wherein the amine masking reduces the formation of secondary structure;
R³ represents an amine masking group; and
B represents a nitrogenous heterocycle.

17. A method of nucleic acid synthesis comprising
a) providing an initiator sequence;
b) adding extension reagents comprising a 3′-O-reversibly terminated nucleoside triphosphate having an amine-masked nitrogenous heterocycle and a terminal deoxynucleotidyl transferase (TdT) to said initiator sequence to add a single nucleotide to the initiator sequence;
c) removal of the extension reagents;
d) cleaving the 3′-O blocking group from the extended nucleic acid polymer;
e) adding extension reagents comprising a 3′-O-reversibly terminated nucleoside triphosphate having nitrogenous heterocycles with free amine groups and a terminal deoxynucleotidyl transferase (TdT) to said initiator sequence to add a single nucleotide to the initiator sequence;
f) removal of the extension reagents;
g) cleaving the 3′-O blocking group from the extended nucleic acid polymer;
h) repeating steps (b)-(g) to produce an extended nucleic acid polymer having a subset of the amine groups masked amine-masked nitrogenous heterocycles; and
i) releasing the NH₂ groups on the nitrogenous heterocycles by removing the amine-masks.

18. A method of nucleic acid synthesis according to claim 17 comprising:
(a) providing an initiator sequence;
(b) adding extension reagents comprising a 3′-O—NH₂ blocked or a 3′-O—CH₂N₃ blocked nucleoside triphosphate having an amine-masked nitrogenous heterocycle and a terminal deoxynucleotidyl transferase (TdT) to said initiator sequence to add a single nucleotide to the initiator sequence;
(c) removal of the extension reagents;
(d) cleaving the 3′-O blocking group from the extended nucleic acid polymer;
(e) adding extension reagents comprising a 3′-O—NH₂ blocked or a 3′-O—CH₂N₃ blocked nucleoside triphosphate having nitrogenous heterocycles with free amine groups and a terminal deoxynucleotidyl transferase (TdT) to said initiator sequence to add a single nucleotide to the initiator sequence;
(f) removal of the extension reagents;
(g) cleaving the 3′-O blocking group from the extended nucleic acid polymer;
(h) repeating steps (b)-(g) to produce an extended nucleic acid polymer having a subset of the amine groups masked amine-masked nitrogenous heterocycles; and
(i) releasing the NH₂ groups on the nitrogenous heterocycles by removing the amine-masks.

19. The method according to claim 18 wherein the synthesised strands are treated with a uracil glycosylase.
